(19) United States
(12) Patent Application Publication
(10) Pub. No.: US 2011/0029010 A1
(43) Pub. Date: Feb. 3, 2011

(54) FLEXIBLE DISSECTING FORCEPS

(76) Inventors: Salvatore Castro, Raleigh, NC (US); Robert E. Welt, Wake Forest, NC (US)

Correspondence Address: SYNECOR LLC, P.O. BOX 5325, LARKSPUR, CA, 94977, US

(21) Appl. No.: 12/511,053

(22) Filed: Jul. 28, 2009

(51) Int. Cl. A61B 17/28

(52) U.S. Cl. 606/206

(57) ABSTRACT

A medical dissector having an elongate flexible shaft and dissecting jaws. The jaws moveable from opened to closed and from closed to open using pull actuation of an elongate actuation members such as a pull cables. A first pull cable controls opening of the jaws, while a second pull cable controls closing of the jaws. The shaft and jaws of the instrument are axially rotatable relative to the handle to rotate the jaws about the longitudinal axis of the shaft.

### RELATED APPLICATIONS

This application claims the benefit of U.S. Application No. 60/145,015, filed Jan. 15, 2009.

### FIELD OF THE INVENTION

The present invention relates generally to the field of medical instruments. More particularly, the present invention relates to flexible dissecting instruments.

### BACKGROUND

An endoscopic surgical dissector is a conventional instrument used for endoscopic procedures, as well as for other procedures such as laparoscopy, single port surgery, and natural orifice procedures. A conventional endoscopic dissector includes a pair of opposed curved jaws for dissecting tissue. The jaws are connected to a common clevis pin mounted in a clevis which is coupled to the distal end of an elongate flexible coil. The coil gives the shaft of the device the requisite flexibility for use in a flexible endoscope or other flexible, articulating or non-linear access device.

At its proximal end, the coil is attached to a handle. The handle contains a spool about which a pull-wire is wound. The pull-wire extends through the coil and is coupled to the jaws. In some conventional dissectors, the handle includes an actuator which is manipulated to close the jaws by pulling on the pull-wire, and to open the jaws by pushing on the pull-wire. When the pull-wire is under tension to close the jaws, the windings of the coil are compressed against one another, allowing axial forces to be transmitted through the coil. However when tension on the pull-wire is released to open the jaws, the coil lacks the column strength for force application to the tissue. Thus, these types of forceps are ineffective for applying forces to tissue (e.g. for spreading of tissue) when the jaws are open. The dissector described in the present application is an improvement over conventional endoscopic graspers, since it allows force to be applied to the tissue whether the jaws are closed or opened.

In other conventional dissectors, the handle actuator opens the jaws by pulling on the pull-wire, and closes the jaws by pushing on the pull-wire. In these embodiments, the closing forces of the jaws may be limited by the stretch of the coil that occurs when the pull-wire is pushed.

### BRIEF DESCRIPTION OF THE DRAWINGS

FIG. 1 is a perspective view of an embodiment of a dissector;

FIG. 2 is a perspective view of the distal end of the dissector of FIG. 1;

FIG. 3A is similar to FIG. 2, but shows the jaws and associated links separated from the shaft and clevis housings;

FIG. 3B is similar to FIG. 3A, but shows the jaws in the closed position and further shows the two devises separated from another and from the coil;

FIG. 3C is an elevation view along the longitudinal axis of the jaw-closing clevis and the jaw-opening clevis, showing the attachment bores for the pull-wires;

FIG. 4 is a side elevation view of the jaws;

FIG. 5 is similar to FIG. 4, but shows the jaws separated from one another;

FIG. 6A is a perspective view of the jaws with the opening clevis and associated links;

FIG. 6B is similar to FIG. 6A, but shows the links exploded from the jaws;

FIG. 7A is a perspective view of the jaws with the closing clevis and associated links;

FIG. 7B is similar to FIG. 7A but shows the link assembly separated from the jaws;

FIG. 7C is similar to FIG. 2, but omits the jaw-opening clevis and the coil;

FIG. 8 is a side view of the handle of the dissector of FIG. 1 with one side of the rotation cap and the proximal housing;

FIG. 9 is a perspective view of the actuator of the handle of FIG. 8;

FIG. 10 is a partially exploded view of the pulley housing, guide shaft, pulley, cables and associated features removed from the handle shown in FIG. 8;

### DETAILED DESCRIPTION

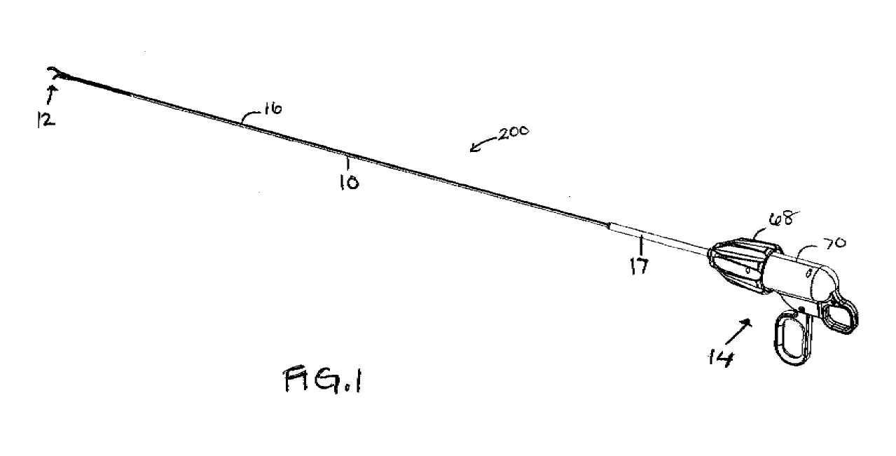

FIG. 1 shows an embodiment of a flexible dissecting forceps device or dissector 200. The dissector 200 includes an elongate shaft 10 having jaws 12 at its distal end. A handle 14 is mounted to the proximal end of the elongate shaft 10. The elongate shaft 10 comprises an elongate coil 16 extending distally from the handle 14. The coil 16 is covered by a flexible outer sleeve formed of a polymeric inner layer, a polymeric outer layer, and a kink-resistant stainless steel braid between the inner and outer layers. A rigid instrument tube 17 is disposed around a proximal portion of the coil 16 and the composite tube in the region of the handle.

Handle 14 includes features for controlling the opening and closing the jaws 12, and for rotating the shaft 10 and jaws 12 about the longitudinal axis of the shaft.

As shown in FIG. 2, a pair of pull-wires, extends through the coil 16. The pull-wires comprise a jaw-closing pull-wire 18a and a jaw-opening pull-wire 18b. A jaw-closing clevis 20 is attached to the distal end of the coil 16 and is coupled to the jaws. A jaw-opening clevis 22 is disposed over the jaw-closing clevis 20 and is slidable relative to the jaw-closing clevis 20 in a longitudinal direction. Upper and lower jaw members 24, 26 extend from the jaw-opening clevis 22.

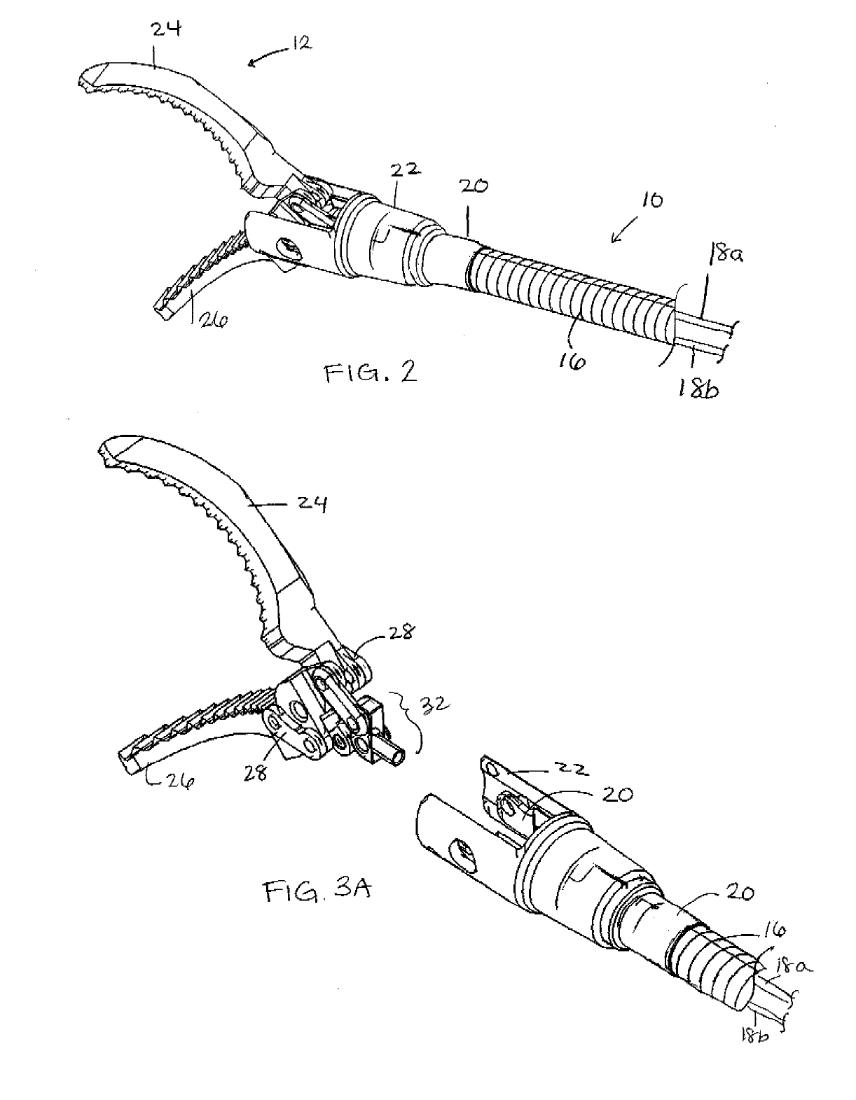

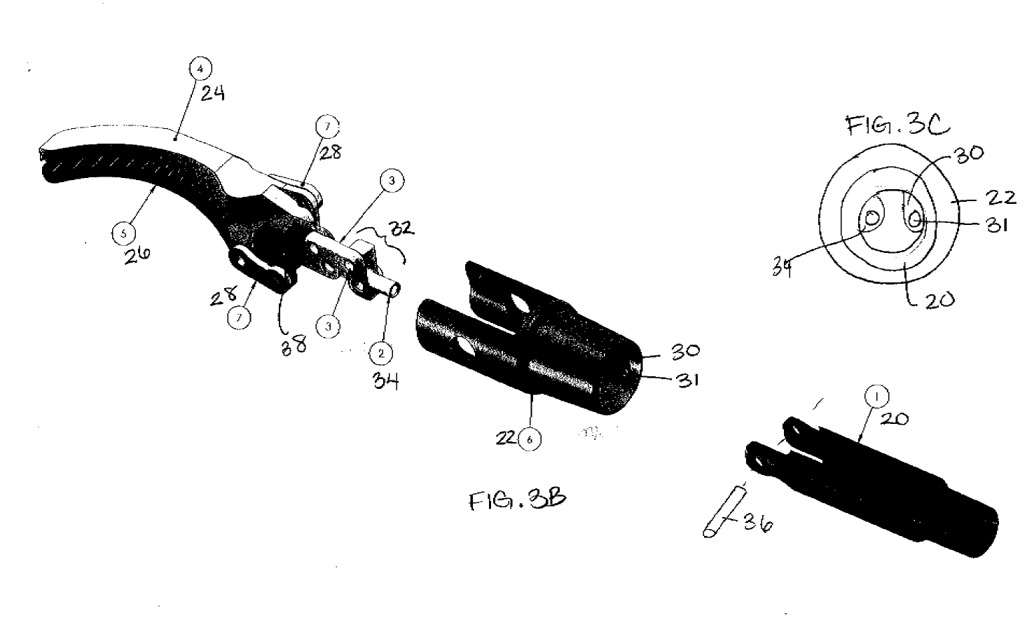

A general overview of the interconnections between the clevises 20, 22, pull wires 18a, 18b and jaw members 24, 26 will be given with reference to FIGS. 3A and 3B. More detailed descriptions will then be given with reference to simplified drawings illustrating these features.

Referring to FIGS. 3A and 3B, links 28 couple the proximal ends of the jaw members 24, 26 to the jaw-opening clevis 22. Machined within the lumen of the jaw-opening clevis 22 is a member 30 (FIG. 3B) used to anchor the jaw-opening pull-wire 18b within the clevis 22. Various methods can be used to couple the pull-wire 18b to the clevis 22, including but not limited to passing the pull-wire 18b through an opening 31 in the member 30 and crimping a cap (not shown) onto the distal end of the wire to prevent it from pulling through the opening 31.

A link assembly 32 couples the proximal ends of the jaw members 24, 26 to the jaw-closing pull-wire 18a. The link assembly includes a tubular extension 34 which is used to anchor the jaw-closing pull wire 18a. As shown in the axial view of FIG. 3C, the axis of the lumen of the tubular extension 34 is laterally offset from the axis of the opening 31 in the member 30, allowing the pull-wires 18a, 18b to extend in parallel through the coil and the devises 30, 32. In the preferred arrangement, the distal termination points of the pull-wires are longitudinally off-set from one another, with the termination of the jaw-opening pull wire 18b at clevis 22 being more proximal than the termination point of the jaw-closing pull-wire 18a at tubular extension 34.

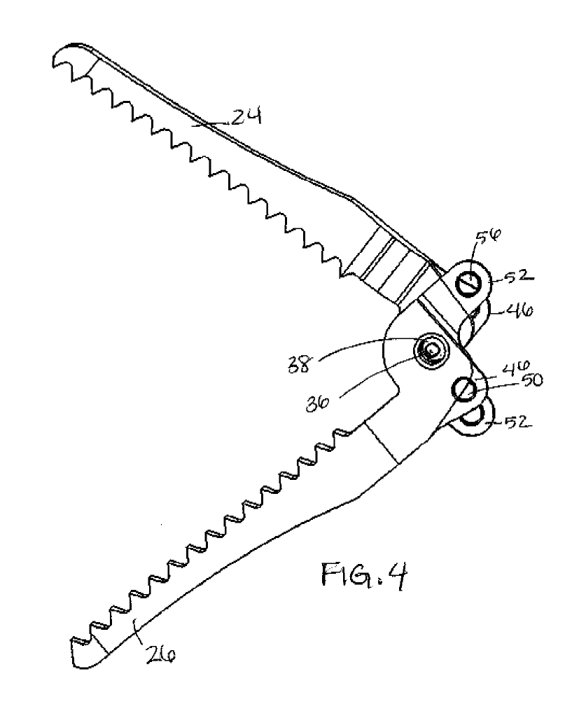

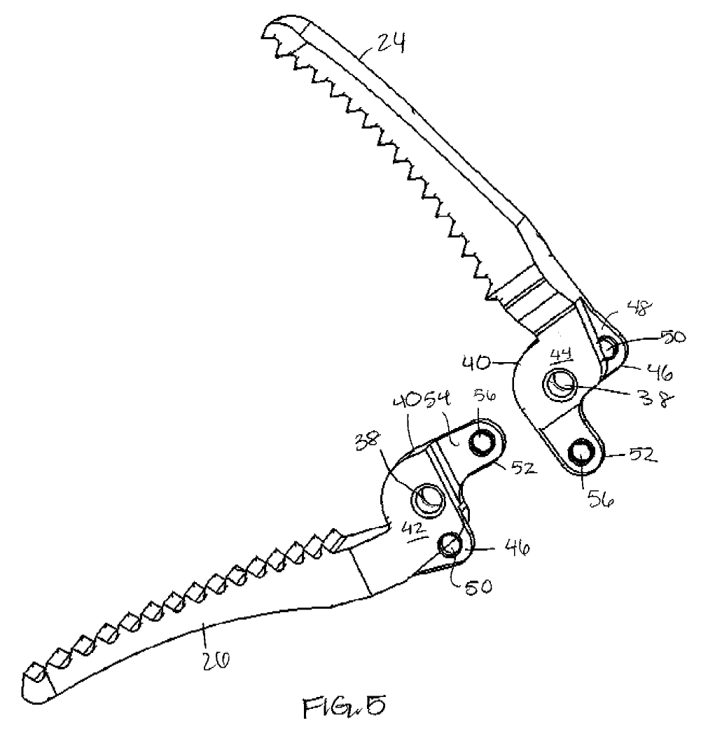

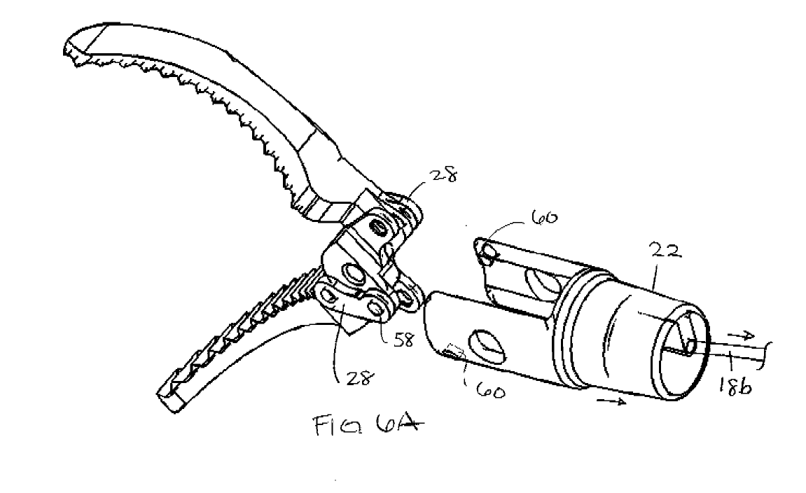

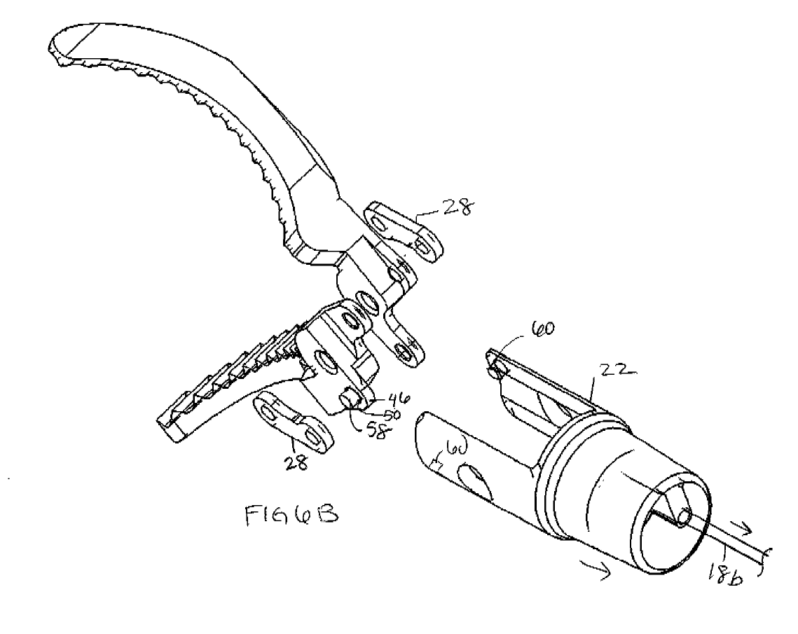

A clevis pin 36 (FIG. 3B) couples the jaw-opening clevis 22 to the jaw members 24, 26. Referring to FIGS. 4 and 5, each of the jaw members 24, 26 has a pivot opening 38 which receives the clevis pin 36. The jaw members 24, 26 are pivotable about the clevis pin 36 when they moved between their open and closed positions.

Each jaw member 24, 26 is an inverted version of the other. The view shown in FIG. 5 shows the laterally outwardly facing side of the lower jaw member 26, and the laterally inwardly facing side of the upper jaw member 24. The proximal ends of each jaw member include a wall 40 having an outer face 42 and an inner face 44. The pivot opening 38 extends through the wall 40 between the faces 42, 44. Wall 40 includes a first tab or tang 46 having an outer surface that is continuous with the outer face 42. The portion of the wall defining the tab 46 is thinner than the full-thickness portion of the wall that extends between the outer and inner faces 42, 44, and thus the tab 46 has an inner surface 48 that is recessed from the inner face 44. A second opening 50 is positioned partially on the tab 46, such that a portion of the opening 50 extends through the tab 46, and the remaining portion is eclipsed by the full thickness part of the wall 40. In alternate embodiments, the opening 50 may simply be a recess in the tab 46.

Wall 40 includes a second tab or tang 52 that extends in a proximal direction. The tab 52 has an inwardly-facing surface that forms a continuous planar surface with the inner face 44 of the wall 40. However, the tab 52 is formed of a thinner section of material than the full-thickness section of the wall 40, and thus its outwardly facing surface 54 is recessed from the outer face 42. A third opening 56 is formed through the tab 52.

Referring again to FIG. 4, the jaw members 24, 26 are arranged in the dissector such that when the opening pull wire (not shown) is pulled, forces are imparted to the jaws at the tabs 46 to pivot the jaws from the closed position (FIG. 3B) to the open position shown in FIG. 4. When the closing pull wire (not shown) is pulled, forces are imparted to the jaws at the tabs 52 to pivot the jaws from the open position to the closed position.

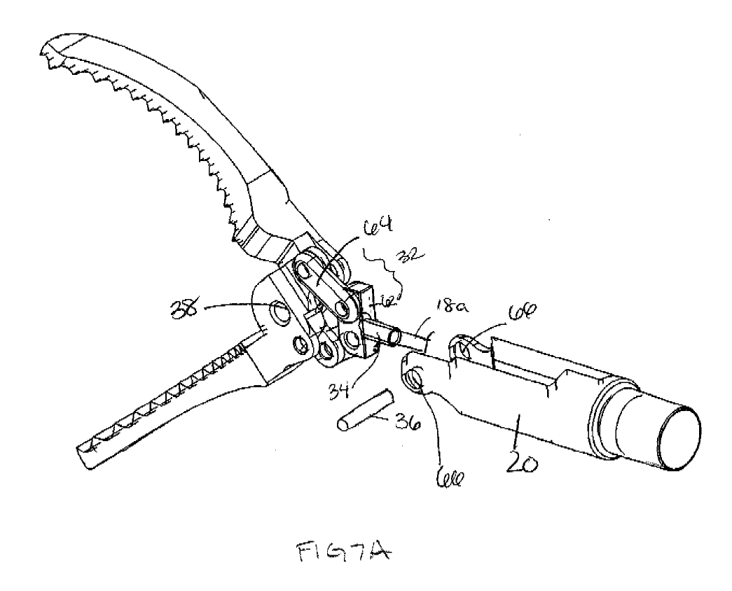

The arrangement of links 28 used to move the jaws to the open position is shown in FIGS. 6A and 6B. The proximal end of each of the links 28 is coupled to the tab 46 by a pin 58 in the opening 50. The distal end of each link 28 is attached to the distal end of the jaw-opening clevis 22 by a second pin 60. When the jaw-opening pull-wire 18b is pulled proximally, the jaw-opening clevis 22 slides proximally relative to the coil 16 (not shown). The corresponding movement of the links 28 pivots the jaw members 24, 26 relative to the clevis pin 36 (FIG. 4), thereby pivoting the jaws to the open position.

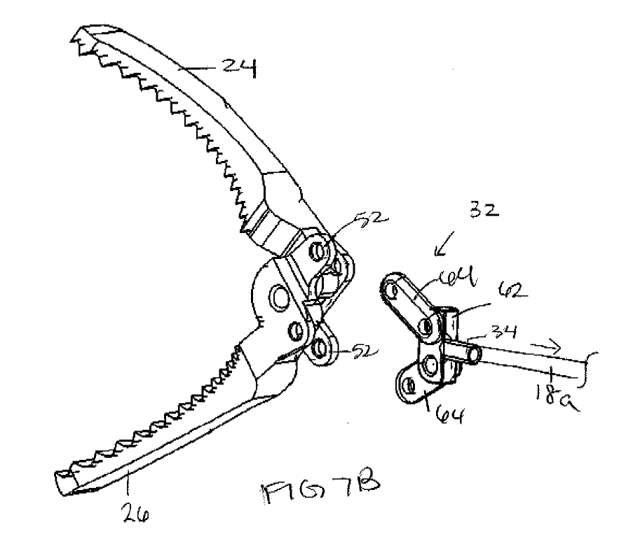

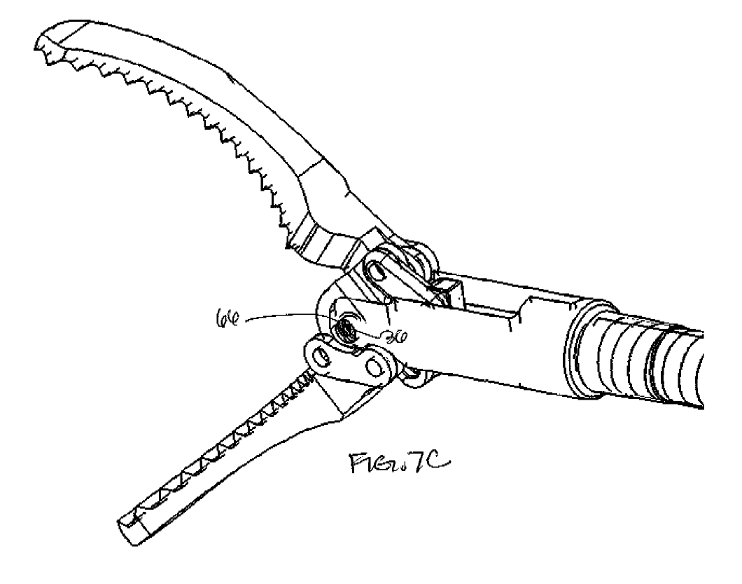

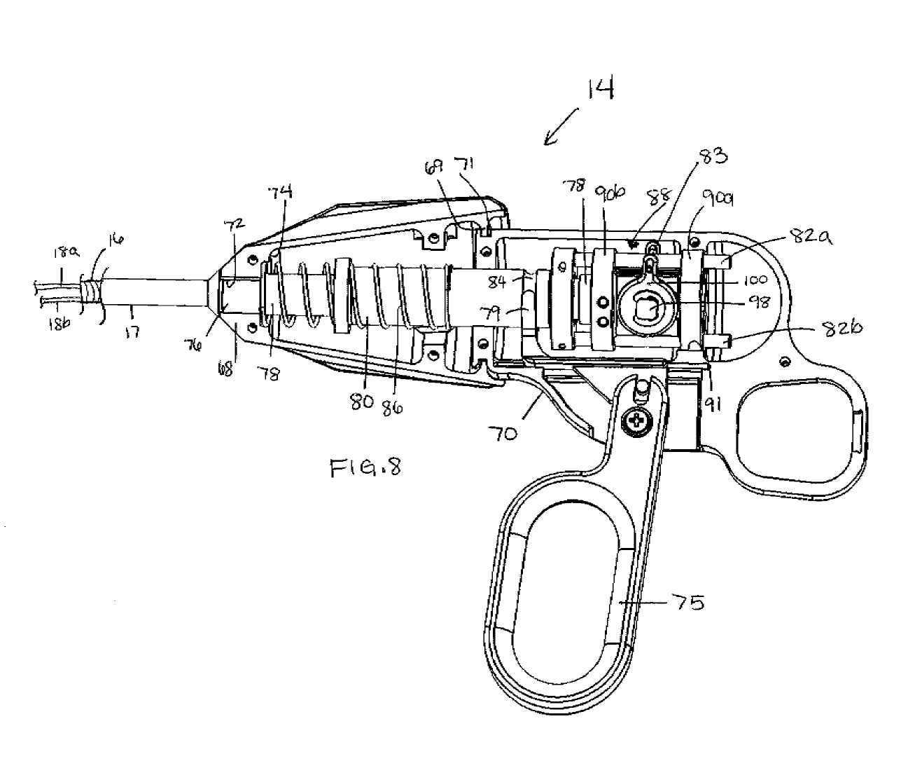

It should be noted that, for clarity, FIGS. 6A and 6B omit the clevis and link elements that are used to close the jaws. FIGS. 7A and 7B feature the jaw-closing features, and omit the jaw-opening features for purposes of clarity. As shown, the jaw-closing features include the link assembly 32, which comprises the actuation tip 62 and the tubular extension 34. Links 64 extend distally from each side of the actuation tip 62. Each of the links 64 is coupled to the tab 52 of a corresponding one of the jaw members 24, 26. The jaw-closing clevis 20 includes distal openings 66 as shown in FIG. 7A. FIG. 7C shows that when the system is assembled, clevis pin 36 extends through these distal openings 66 and through the corresponding pivot openings 38 in the jaw members 24, 26. The jaw-closing pull-wire 18a extends from the coil 16 (not shown) into the jaw-closing clevis 20 and is anchored to the tubular extension 34 of the actuation tip 62 as shown in FIG. 7B.

During use, pulling on the jaw-closing pull-wire 18a pulls the actuation tip 62 proximally relative to the jaw-closing clevis 20, causing the links 64 to pull the tabs 52 proximal, thereby pivoting the jaws 24, 26 relative to the clevis pin 36 and causing the jaws the close.

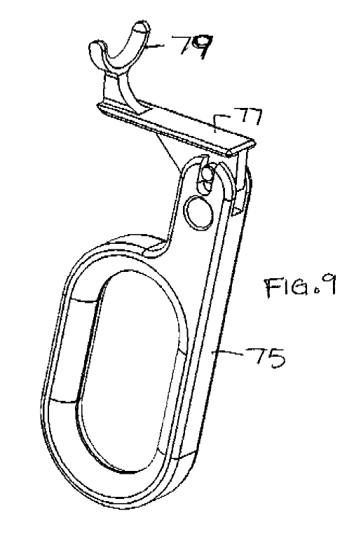

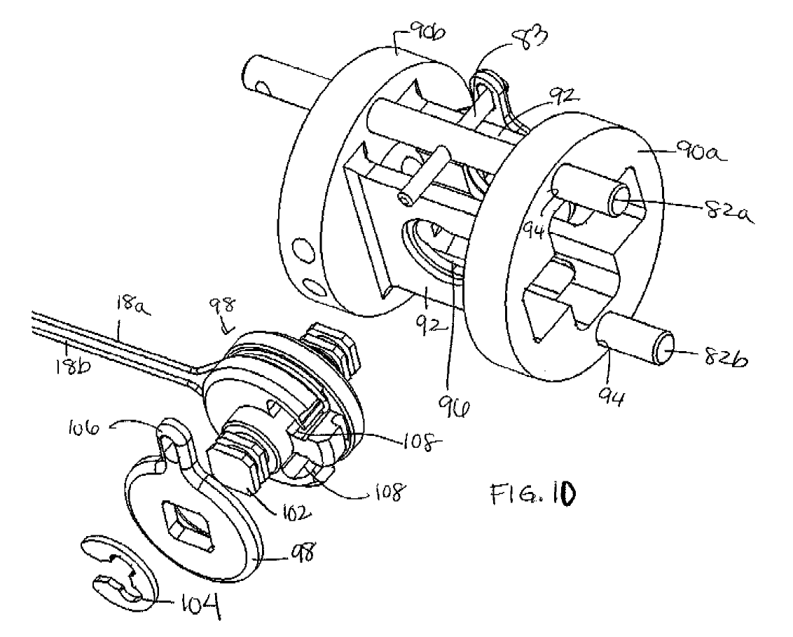

Referring again to FIG. 1, the handle 14 includes a nose piece 68 and a proximal handle section 70. FIG. 8 illustrates the handle 14 with one half of the nose piece 68 and one half of the proximal handle section 70 removed to reveal the internal features. As shown, the nose piece 68 is a hollow cap having a tapered distal end having a bore 72. An annular wall section 74 surrounds the bore 72 within the hollow interior of the nose piece 68. The proximal end of the nose piece 68 is open and mates with the distal end of the proximal handle section 70. A circumferential rib 69 extends radially inwardly near the proximal opening.

The proximal handle section 70 is a hollow housing having a distal opening. A circumferential groove 71 extends around the proximal handle section near the distal opening. When the handle is assembled, the circumferential rib 69 of the nose piece 68 is positioned within this groove 71. Proximal handle section 70 also includes a finger grip 73 and an actuator such as a finger pull 75 pivotally mounted adjacent to the finger grip. The actuator includes a sliding link 77 that is cammed in a longitudinal direction by the pivoting motion of finger pull 75. In the disclosed embodiment, an arcuate member 79 is carried by the sliding link 77.

Referring again to FIG. 8, a transition tube 76 is seated within the bore 72. The instrument tube 17 and the coil extending through it (not shown) extend into, and terminate within, the transition tube 76. The transition tube 76 extends into and terminates with a second tube 78. The second tube 78 extends into the hollow interior of the proximal handle section 70. An interface cylinder 80 is telescopingly received over the second tube 78 and it is longitudinally slidable relative to the second tube 78. Interface cylinder 80 has a proximal portion which extends the length of nose piece 68 and into the proximal handle section 70. Upper and lower rods 82a, 82b are attached to the proximal end of the interface cylinder and cantilever in a proximal direction. A dowel 83 extends perpendicularly through upper rod 82a. A circumferential groove 84 is formed on the exterior surface of the interface cylinder 80, preferably near its proximal end. Arcuate member 79 of actuator 75 is disposed within the groove 84.

A coil spring 86 has a proximal end attached to the interface cylinder 80, and a distal end in contact with the annular wall section 74 within the nose piece 68.

A pulley housing 88 is mounted within the proximal housing section 70. As shown in FIG. 10, pulley housing 88 includes proximal and distal plates 90a, 90b and side walls 92 extending between the plates 90a, 90b. The distal plate 90b is mounted to the proximal end of the second tube 78 as shown in FIG. 8. The proximal plate 90a is positioned in contact with a feature within the proximal housing section 70, such as rib 91. Upper and lower openings 94 are formed in the plates 90a, 90b and are slidably disposed over the parallel rods 82a, 82b extending from the interface cylinder 80 (FIG. 8). Cutouts 96 are formed in the side walls 92.

A pulley 98 is positioned between the plates 90a, 90b and the side walls 92 of the pulley housing 88. The pulley 98 includes hubs 102 that extend through the cutouts 96 in the side walls 92. Links 100 are positioned on the outer surfaces of the side walls 92 of the pulley housing and are coupled to the hubs 102 by retaining rings 104. Each link 100 includes an element 106 attached to the dowel 83 as shown.

The pull-wires 18a, 18b extend through the coil 16, instrument tube 17 and second tube 78. The proximal portions of the wires are spooled around the pulley 98 as shown in FIG. 10. In the illustrated embodiment, the jaw-closing pull-wire 18a extends around the bottom of the pulley 98 and the jaw-opening pull-wire 18b extends over the top of the pulley 98. The distal ends of the pull-wires 18a, 18b are anchored within slots 108 inside the pulley 98 as shown.

Use

To close the jaws, the user squeezes the finger pull 75 towards the finger grip 73. The finger pull 75 pivots relative to the proximal housing section 70, pushing the arcuate member 79 in a distal direction and thereby advancing the interface cylinder 80 distally against the spring 86. Distal movement of the interface cylinder 80 moves the rods 82a, 82b distally, causing the links 100 to be pivoted distally by the pin 83. Distal rotation of the links 100 causes distal rotation of the pulley 98. This places jaw-closing wire 18b under tension, causing the jaws to close by action of the links 28 (not shown) as described above.

To re-open the jaws, the user releases the finger pull 75. The expansion forces of the spring 86 push the interface cylinder 80 proximally, thereby causing the link 100 to be pivoted proximally by the pin 83. Proximal rotation of the link 100 causes proximal rotation of the pulley 98. This places jaw-opening wire 18a under tension, causing the jaws to open by action of the actuation tip 62 as described above.

Regardless of whether the jaws are open or closed, one of the pull-wires 18a, 18b is always under tension. Because of this, the windings of the coil 16 remains sufficiently compressed to give the shaft 10 column strength sufficient to allow the user to impart forces to tissue using the opened or closed jaws. However, the coil construction of the shaft gives the shaft sufficiently flexibility for use in environments requiring flexibility. For example, the dissector may be used to perform procedures through the instrument shaft of a flexible endoscope, or through other types of deflectable access tubes used to introduce the dissector into the body and to deflect the distal end of the dissector inside the body.

The arrangement of the dissector features allows the user to axially rotate the jaws without changing the position of the handle 14. To do this, the user rotates the nose piece 68 relative to the proximal handle section 70. Rotation of the nose piece 68 causes rotation of the coil 16 (which has the jaws 12 mounted to its distal end), and further rotates all of the features within the handle that are used to open and close the jaws. As the components rotate, the interface cylinder 80 rotates relative to the arcuate member 79, with the arcuate member 79 continuing to ride within the circumferential groove of the arcuate member. The proximal plate 90a of the pulley assembly 88 slides over the rib 91 within the proximal handle section 70 during rotation.

While certain embodiments have been described above, it should be understood that these embodiments are presented by way of example, and not limitation. It will be apparent to persons skilled in the relevant art that various changes in form and detail may be made therein without departing from the spirit and scope of the invention. This is especially true in light of technology and terms within the relevant art(s) that may be later developed.

Any and all patents, patent applications and printed publications referred to above are incorporated by reference.

We claim:

1. A medical instrument, comprising:
a flexible elongate tubular shaft including an elongate tubular coil;
a pair of jaw members at a distal end of the elongate shaft, the jaw members moveable between closed and opened positions;
a first actuation member extending through the tubular shaft, the first actuation member operatively coupled to the jaw members such that pulling the first actuation member longitudinally compresses the coil and moves the jaw members to the closed position;
a second actuation member extending through the tubular shaft, the second actuation member operatively coupled to the jaw members such that pulling the second actuation member longitudinally compresses the coil and moves the jaw members to the opened position; and
at least one actuator coupled to proximal portions of the first and second actuation members for selectively pulling the first and second actuation members in a proximal direction to close and open the jaws.

2. The medical instrument of claim 1, wherein the at least one actuator includes a rotatable element, the first and second actuation members including cables connected to the rotatable element such that rotation of the element in a first direction pulls the first cable in a proximal direction, and rotation of the element in a second direction pulls the second cable in a proximal direction.

3. The medical instrument of claim 2 wherein the rotatable element comprises a pulley.

4. The medical instrument of claim 1, further including a handle including a base and a proximal piece rotationally fixed to the elongate shaft, the proximal piece rotatable on the base relative to the longitudinal axis of the shaft to rotate the elongate shaft, jaws, first and second actuation members about the longitudinal axis.

5. The medical instrument of claim 4, wherein:
the actuator includes a first portion and a second portion, the first portion disposed within the handle and being connected to the first and second actuation members, the second portion in contact with the first portion within the handle and extending from the handle for manipulation by a user to a first position to pull the first cable in a proximal direction and to a second position to pull the second cable in a proximal direction; and
the first portion is coupled to the proximal piece of the handle such that rotation of the proximal piece of the handle relative to the base and the second portion.

6. The medical instrument of claim 1, further including:
a first clevis mounted to a distal end of the elongate tubular shaft, the first clevis including a clevis pin coupled to the jaw members;
an actuation linkage including a pair of first links and a head, each of the first links coupled between a corresponding one of the jaw members and the head, the head attached to the distal end of the first actuation member such that pulling of the first action member in a proximal direction moves the head proximally relative to the first clevis, causing the jaw members to pivot about the clevis pin into the closed position;
a second clevis having a lumen slidably disposed over the first clevis and a pair of second links, each of the second links coupled to a corresponding one of the jaws, the second clevis attached to the distal end of the second actuation member such that pulling of the second actuation member in a proximal direction slides the second clevis in a proximal direction relative to the first clevis, causing the jaw members to pivot about the clevis pin into the opened position.

7. The medical instrument of claim 6, wherein each jaw member includes a first tang and a second tang, wherein the first tang is coupled to a corresponding one of the first links and the second tang is coupled to a corresponding one of the second links.

8. A method of operating a medical instrument, comprising:
providing a medical instrument comprising a flexible elongate shaft including an elongate flexible coil, a pair of jaws at the distal end of the shaft, and a pair of cables extending through the shaft;
pulling a first one of the cables to compress the flexible coil and to close the jaws; and
pulling a second one of the cables to compress the flexible coil and to open the jaws.

9. The method of claim 8, wherein the method provides a handle at the proximal end of the instrument and an actuator coupled to the handle, the method include moving the actuator in a first direction to pull the first cable, and moving the actuator in a second direction to pull the second cable.

10. The method of claim 9, where the method provides the instrument to include a pulley within the handle wherein the first and second cables are attached to the pulley, and wherein the method further includes moving the actuator in a first direction to rotate the pulley in a first direction to pull the first cable, and moving the actuator in a second direction to rotate the pulley in a second direction to pull the second cable.

11. The method of claim 10, wherein moving the actuator rotates the pulley in a direction transverse to the longitudinal axis of the flexible elongate shaft.

12. The method of claim 9, further including the step of rotating a rotatable portion of the handle relative to a base portion of the handle, wherein rotating the rotatable portion rotates the flexible elongate shaft, and first and second cables relative to the base portion.
